(19) United States
(12) Patent Application Publication
(10) Pub. No.: US 2009/0275814 A1
(43) Pub. Date: Nov. 5, 2009

(54) SYSTEM AND METHOD FOR MEASURING CONSTITUENT CONCENTRATION

(75) Inventors: Shinsuke Watanabe, Tokyo (JP); Akira Inoue, Tokyo (JP); Hiroshi Yoshida, Osaka (JP)

Correspondence Address: LEYDIG VOIT & MAYER, LTD, 700 THIRTEENTH ST. NW, SUITE 300, WASHINGTON, DC, 20005-3960, US

(73) Assignees: Mitsubishi Electric Corporation, Tokyo (JP); Nipro Corporation, Osaka-shi, Osaka (JP)

(21) Appl. No.: 12/302,187

(22) PCT Filed: Jun. 8, 2007
(86) PCT No.: PCT/JP2007/061631

§ 371 Date: Nov. 24, 2008

(30) Foreign Application Priority Data
Jun. 12, 2006 (JP) 2006-162391

(51) Int. Cl. A61B 5/1455

(52) U.S. Cl. 600/316; 600/310

(57) ABSTRACT

A system and method for measuring concentration of a constituent of a specimen are provided. The system includes an oscillator for outputting, towards the specimen, electromagnetic waves having respective different frequencies between 5 GHz and 300 GHz; a detector for detecting the electromagnetic waves that are reflected from the specimen; and a processor measuring at least one of reflection coefficient and complex permittivity of the electromagnetic waves detected and calculating the concentration of the constituent of the specimen based upon at least one of the reflection coefficient measured and the complex permittivity measured.

### TECHNICAL FIELD

The present invention relates to a system and method for measuring a concentration of desired constituent of a specimen.

### BACKGROUND ART

Diabetes is an adult disease, rapidly increasing the serum glucose concentration (blood-sugar level) caused by reduced output of insulin, which often suffers complications such as cardiovascular disorder, cerebral infarction, foot sphacelus, and blindness by retinodialysis. The Ministry of Health, Labour and Welfare of Japan has announced, according to an actual survey of the diabetes in 2002, that about 7.4 million people are “highly suspected”, and about 16.2 million people (i.e., one in about 6.3 Japanese) are undeniably suspected to suffer the diabetes. It is predicted that the number of patients suffering the diabetes is still increasing not only in Japan but also worldwide. Also, since the diabetes itself is an asymptomatic disease until suffering extreme blood-sugar level or serious complications, it is particularly important to have a routine medical check including the blood test for early diagnosis, thereby preventing the diabetes.

The blood test is typically used for monitoring the blood-sugar level in real-time, which requires stinging a needle into the patient's skin and sampling the patient's blood therethrough. However, this blood test inflicts much pain on the patient and raises possible risks of infections to the others unless the needle is safely disposed. Therefore, it has highly been desired to develop a non-invasive approach for precisely measuring the serum glucose concentration, without sampling the blood.

Several non-invasive approaches for measuring the serum glucose concentration have been proposed so far. For example, the Japanese Patent Publication No. 2005-237867 (Patent Document 1) discloses a system and method for measuring the blood-sugar level by means of near infrared rays. The serum glucose resonates with and absorbs the near infrared rays of particular wavelengths, caused by stretching and bending of bindings between atoms composing the glucose such as hydrogen, carbon, nitrogen and oxygen. In accordance with this knowledge, Patent Document 1 discloses the system and method for measuring the blood-sugar level, which illuminates the near infrared rays of particular wavelengths on the specimen and measures the absorption level thereof, thereby to determine the glucose concentration.

Besides the near infrared rays as suggested by Patent Document 1, another approach using a millimeter wave has also been proposed for measuring the blood-sugar level. For example, the Japanese Patent Publication No. 2006-000659 (Patent Document 2) discloses a non-invasive system and method for measuring the blood-sugar level by means of the millimeter wave. In general, since sugars contain many functional groups causing hydrogen bonding (typically hydroxyl group) per unit mass, the dielectric constant of water may likely be variable with sugars added therein. Therefore, the non-invasive system of Patent Document 2 illuminates the millimeter wave of single wavelength on the measured dielectric sample such as blood sample and is designed to minimize a reflection coefficient of single millimeter wave at a given wavelength reflected at the measured dielectric sample, over the measured spectrum. This allows measurement of the serum glucose concentration based upon the corresponding minimum frequency and measured temperature of the dielectric sample to be measured.

Also, another technique of the non-invasive blood-sugar measurement with the millimeter wave is suggested in the article of “Collected Papers, Electronic I, 2001, page 164, by Institute of Electronics, Information and Communication Engineers, (Non-patent Document 1)”. The Non-patent Document 1 teaches measurement of the permeability coefficient of glucose aqueous solution added with sodium chloride, by illuminating the millimeter wave onto the solution, and concludes frequency dependency of the permeability coefficient in accordance with different glucose concentrations.

### Patent Document 1: JPA 2005-237867

### Patent Document 2: JPA 2006-000659

Non-patent Document 1: “Collected Papers, Electronic I, 2001, page 164, by Institute of Electronics, Information and Communication Engineers”

### DISCLOSURE OF INVENTION

### Problems to be Solved by Invention

However, according to the measuring system of the blood-sugar level by means of near infrared rays as described in Patent Document 1, since the blood, in fact, contains various components other than the glucose, having the bindings between atoms such as hydrogen, carbon, nitrogen and oxygen, it is practically difficult to determine the glucose concentration based upon absorption of the near infrared rays of particular wavelengths.

Also, according to the non-invasive measuring system of the blood-sugar level by means of the millimeter wave as suggested in Patent Document 2, since reflection coefficient (i.e., dielectric constant) may be varied based upon concentrations of not only glucose but also other components such as albumin and hemoglobin, the concentration of glucose cannot precisely be measured.

Therefore, one of embodiments according to the present invention addresses the aforementioned drawbacks, and has a purpose to provide a non-invasive system and method for precisely measuring concentration of desired constituent of a specimen, for example, glucose concentration of a blood.

### Means to Solve the Problems

The present inventors has discovered that the desired constituent of a specimen can sophisticatedly be determined by measuring a reflection coefficient or complex permittivity of the electromagnetic waves at two or more frequencies, particularly noting that the measured reflection coefficient (reflection power and reflection phase) and the complex permittivity have frequency dependency affected by the concentrations of various constituents in the specimen, such as glucose, albumin and hemoglobin.

Therefore, one of aspects of the present invention is to provide a system and method for measuring a concentration of desired constituent of a specimen. The system includes an oscillator for outputting towards the specimen, a plurality of electromagnetic waves having frequencies between 5 GHz and 300 GHz that are different from one another. It also includes a detector for detecting the electromagnetic waves reflected at the specimen. Further it includes a processor for measuring at least either one of a reflection coefficient and a complex permittivity for the electromagnetic waves and calculating the concentration of the desired constituent of the specimen based upon the at least either one of the reflection coefficient and the complex permittivity.

### ADVANTAGES OF INVENTION

One of aspects of the present invention provides a non-invasive system and method for precisely measuring concentration of desired constituent of the specimen.

### BRIEF DESCRIPTION OF DRAWINGS

FIG. 1 is a schematic view illustrating a first embodiment of a measuring system according to the present invention.

FIG. 2 is a block diagram illustrating components of the measuring system shown in FIG. 1.

FIGS. 3A and 3B are charts showing frequency dependency of the reflection power (Γ) and reflection phase (Φ) of millimeter waves reflected at a blood, respectively, varying with frequency of the millimeter waves.

FIG. 4 is a schematic view illustrating a measuring system of Modification 2.

FIG. 5 is a schematic view illustrating a measuring system of Modification 3.

FIG. 6 is a schematic view illustrating a cavity resonator of Modification 4.

FIGS. 7A and 7B are charts showing frequency dependency of real and imaginary parts of the complex permittivity, respectively, for a blood containing different serum glucose concentrations, varying with frequency of the millimeter waves.

FIGS. 8A and 8B are charts showing frequency dependency of real and imaginary parts of the complex permittivity, respectively, of a blood containing different sodium chloride concentrations, varying with frequency of the millimeter waves.

FIGS. 9A and 9B are charts illustrating a plurality of measured points (with dots) of real and imaginary parts of the complex permittivity of millimeter waves reflected at a specimen, respectively, and trajectories thereof (with a line) continuously approximated by a dielectric relaxation equation.

FIGS. 10A and 10B are charts showing frequency dependency of real and imaginary parts of the complex permittivity, respectively, for a blood containing glucose and hemoglobin, varying with frequency of the millimeter waves.

### BRIEF DESCRIPTION OF REFERENCE NUMERALS

1, 1′, 1″, 2: measuring system
10: oscillator
12, 14: oscillating member
13, 15: phase-synchronizing loop circuitry
16: voltage-control oscillating element
17: internal oscillating element
18: frequency dividing element
19: phase comparing element
20: detector
22: coupler
24: circulator
23, 25: frequency dividing element
26: amplitude comparator
28: phase comparator
30: oscillation-detection apparatus
40: cavity resonator
42, 43: chassis,
44: coaxial cable
45: dielectric rod
46, 48: dielectric material
47: phase shifter
50: processor
60: thermal sensor

### BEST MODE FOR CARRYING OUT THE INVENTION

Described herein with reference to attached drawings are several embodiments of a system for measuring concentration of desired constituent of specimen, according to the present invention.

### Embodiment 1

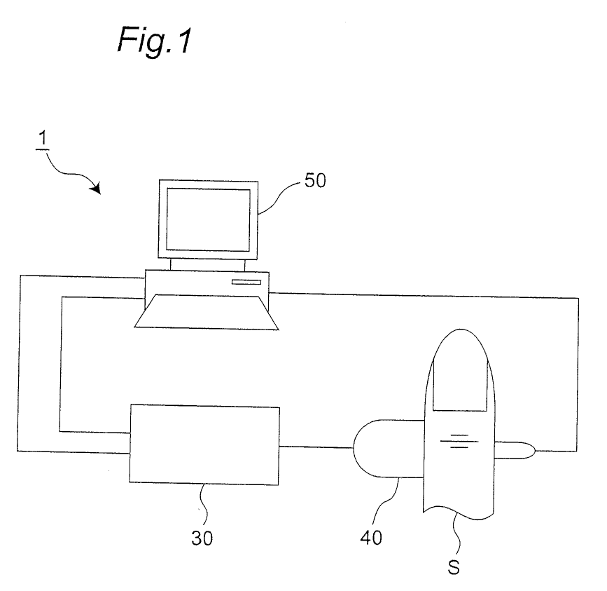

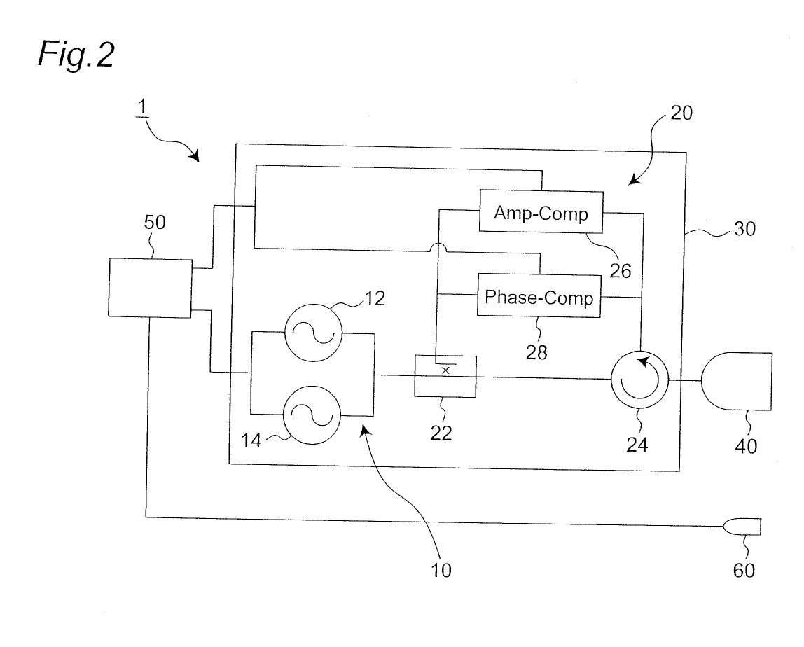

FIG. 1 is a schematic view illustrating a first embodiment of a measuring system according to the present invention. Also, FIG. 2 is a block diagram illustrating components of the measuring system shown in FIG. 1. The measuring system 1 of FIGS. 1 and 2 generally includes an oscillation-detection apparatus 30, which includes an oscillator 10 outputting an electromagnetic wave having variable frequency between 5 GHz and 300 GHz, towards a specimen (test body) S such as user's finger, and a detector 20 detecting the electromagnetic wave reflected at the specimen S. Also, the measuring system 1 includes a cavity resonator 40 contacting with the specimen S, which is connected to the oscillation-detection apparatus 30, and a processor 50 such as a personal computer for driving the oscillator 10 of the oscillation-detection apparatus 30 and also for processing data signals from the detector 20. Further, the measuring system 1 preferably includes a thermal sensor 60 for measuring temperature of the specimen S. It should be noted that the electromagnetic waves having frequencies in the range between 3 GHz and 30 GHz and between 30 GHz and 300 GHz are generally referred to as “centimeter wave” and “millimeter wave”, respectively. Therefore, the electromagnetic wave having frequency in the range between 5 GHz and 300 GHz will be referred hereinafter to as “semi-millimeter wave or millimeter wave” or simply as “centi-millimeter wave”.

As illustrated in FIG. 2, the oscillator 10 of the oscillation-detection apparatus 30 includes first and second oscillating members 12, 14 outputting first and second centi-millimeter waves having first and second frequencies (f₁=26.4 GHz, f₂=30.9 GHz), respectively. The first and second centi-millimeter waves are transmitted through a coupler 22 and a circulator 24 to the cavity resonator 40, in which the waves are caused to be resonated. Then, the first and second centi-millimeter waves resonated in the cavity resonator 40 reflect at the blood (the blood containing various constituents such as glucose, albumin, and hemoglobin) running close to the surface of the test body such as user's finger, and back to the cavity resonator 40. The first and second centi-millimeter waves returned to the cavity resonator 40 are transmitted through the circulator 24 of the oscillation-detection apparatus 30 to the detector 20.

As illustrated in FIG. 2, the detector 20 of the oscillation-detection apparatus 30 includes an amplitude comparator 26 and a phase comparator 28 connected directly with the coupler 22 and the circulator 24.

The amplitude comparator 26 compares the voltage amplitude of the first and second centi-millimeter waves output from the oscillator 10 (input voltage Vᵢₙ) with those reflected at the specimen S (output voltage Vₒᵤₜ), and the processor 50 calculates the reflection powers (Γ₁, Γ₂) which are decibel-converted by the following equations.

$${Γ}_{1} = 20 \times log ( {V}_{out 1} / {V}_{in 1} )$$

$${Γ}_{2} = 20 \times log ( {V}_{out 2} / {V}_{in 2} ) [ unit \text{:} dB ]$$

Similarly, the phase comparator 28 detects phase shifts (reflection phases) between the first and second centi-millimeter waves output from the oscillator 10 and those reflected at the specimen S, generating phase shift signals which are transmitted to the processor 50.

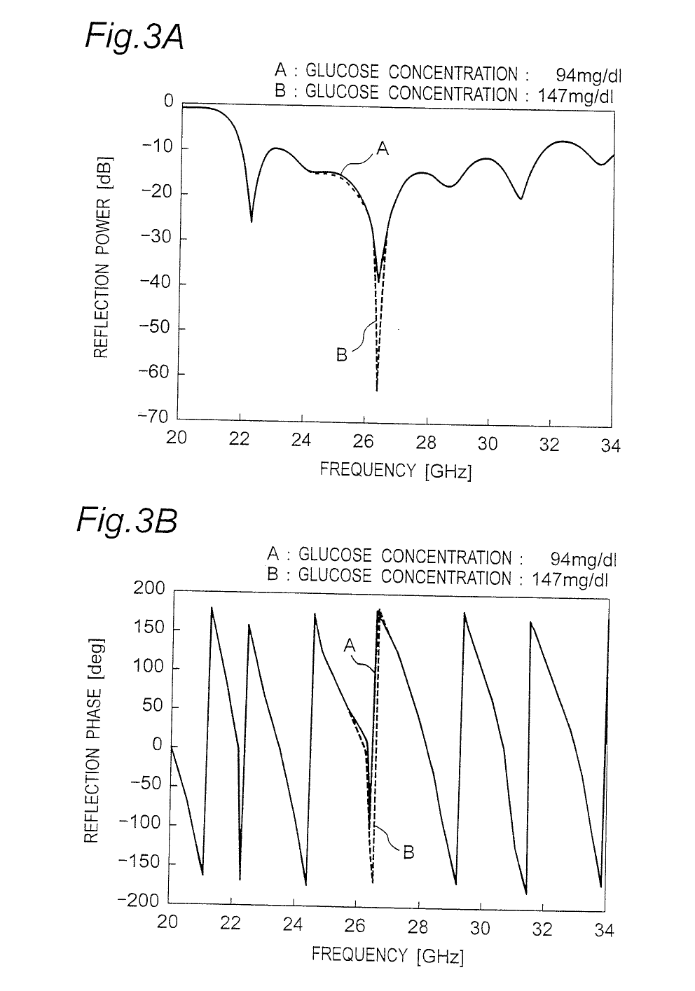

In the meanwhile, as illustrated in FIGS. 3A and 3B, the reflection power (Γ) and the reflection phase (Φ) are varied in accordance with frequency of the centi-millimeter wave, respectively, and strongly affected by the serum glucose concentration with the frequency especially around 26.4 GHZ. For this reason, the conventional non-invasive blood-sugar measuring system adapts the semi-millimeter wave or millimeter wave for estimating an unknown blood-sugar level (BS) for the measured reflection power (Γ) based upon the known relationship between the blood-sugar level (BS) and the reflection power (Γ).

In particular, a correction function, as expressed below, of a quadratic equation with one unknown parameter of the measured reflection power (Γ) is firstly presumed for determining the blood-sugar level (BS), and the factors of the correction function are empirically calculated based upon the measured values of the reflection powers (Γ) for the known blood-sugar levels (BS) at frequency of 26.4 GHz (i.e., based upon the relation therebetween). As above, since the reflection power (Γ) and the reflection phase (Φ) are strongly affected by the serum glucose concentration with the centi-millimeter wave having the frequency at 26.4 GHZ, the conventional system uses a single centi-millimeter wave having this particular frequency illuminated onto the specimen so as to measure the reflection power (Γ), thereby calculating the serum glucose concentration (blood-sugar level) in the specimen though the following equation.

BS=p×Γ²+q×Γ+r

The factors p, q, r are empirically determined as 5.43×10⁻², 7.55, and 354, respectively.

However, the reflection power (Γ) may be affected by not only the glucose concentration but also other blood constituent concentrations. Therefore, the blood-sugar level estimated by assigning the measured reflection power (Γ) into the above equation, may often be inconsistent with the actual measurement as indicated below.

TABLE 1

| Measured | Estimated | Measured |
|---|---|---|
| Reflection Power (Γ) | Blood Sugar Level | Blood Sugar Level |
| −42.94 dB | 130 mg/dl | 140 mg/dl |

To address the deficiency, another trial is made, presuming a different correction function, as expressed below, of a quadratic equation with two unknown parameters including the measured reflection power (Γ) and the reflection phase (Φ) for determining the blood-sugar level (BS). Then, each of the factors in the correction function is calculated based upon the measured values of the reflection power (Γ) and the reflection phase (Φ) for the known blood-sugar levels (BS).

BS=p×Γ²+q×Γ+r×Φ²+s×Φ+t,

wherein the factors p, q, r, s, and t are constants. However again, it has been proved that this correction function with two unknown parameters (Φ in addition to Γ) cannot sufficiently remove an influence of the other blood constituent concentrations.

Thus, according to the first embodiment of the measuring system and the measuring method according to the present invention, as described above, the oscillation-detection apparatus 30 illuminates towards the specimen (test body), the centi-millimeter waves having frequencies different from each other (f₁=26.4 GHz, f₂=30.9 GHz), and the processor 50 detects, for each of the centi-millimeter waves, the reflection powers (Γ₁, Γ₂) and the reflection phase (Φ₁, Φ₂) of the specimen. Also, the present invention defines a new correction function expressed in a form of a quadratic equation with four unknown parameters including the measured reflection powers (Γ₁, Γ₂) and the reflection phases (Φ₁, Φ₂) at two different frequencies, and then calculates each of the factors in this correction function based upon the measured values of the reflection powers (Γ₁, Γ₂) and the reflection phases (Φ₁, Φ₂) for the known blood-sugar levels (BS). (Thus, the relationship between the blood-sugar levels (BS) and the parameters, i.e., the reflection powers (Γ₁, Γ₂) and the reflection phases (Φ₁, Φ₂), is empirically calculated.) Therefore, according to the first embodiment, the glucose concentration can be estimated in a quite precise manner by illuminating the first and second centi-millimeter waves having frequencies different from each other to determine the reflection powers (Γ₁, Γ₂) and the reflection phases (Φ₁, Φ₂) of the specimen, and by assigning those four valuables into the new correction function as expressed below.

BS=p₁×Γ₁²+q₁×Γ₁+r₁×Φ₁²+s₁×Φ₁+p₂×Γ₂²+q₂×Γ₂+r₂×Φ₂²+s₂×Φ₂+t

wherein the factors of the correction function are calculated as follows.
p₁=−1.27×10⁻², q₁=−1.27×10⁻²,
r₁=−5.36×10⁻⁴, s₁=1.90×10⁻¹,
p₂=1.17×10⁻², q₂=−3.43×10⁻³,
r₂=4.04×10⁻², s₂=−9.31×10⁻³, t=3.14×10⁻⁴

As an example, the reflection powers (Γ₁, Γ₂) and the reflection phases (Φ₁, Φ₂) of the specimen are actually measured and assigned into the above correction function to estimate the blood-sugar level (BS). It is confirmed as shown below, the estimated blood-sugar level is consistent satisfactorily enough with the real measured blood-sugar level.

TABLE 2

| Reflection | Reflection | Reflection | Reflection | Estimated | Measured |
|---|---|---|---|---|---|
| Power | Phase (φ₁) | Power (Γ₂) | Phase (φ₂) | Blood Sugar | Blood |
| (Γ₁) (26.4 GHz) | (26.4 GHz) | (30.9 GHz) | (30.9 GHz) | Level | Sugar Level |
| −42.83 dB | 259.50 deg | −19.93 dB | −59.70deg | 141 mg/dl | 140 mg/dl |

It should be noted that the reflection powers (Γ₁, Γ₂) and the reflection phases (Φ₁, Φ₂) depend on temperature of the specimen, that is, the estimated blood-sugar level (BS) may vary with temperature of the specimen. Therefore, a set of the correction function factors may preferably be predefined for various thermal points and stored as a table in a memory (not shown) in the processor 50. As described above, the blood-sugar level (BS) can be precisely estimated (measured) without influence of the other constituent concentration, by outputting the centi-millimeter waves having frequencies different from each other to determine the reflection powers (Γ₁, Γ₂) and the reflection phases (Φ₁, Φ₂) of the specimen.

Modification 1.

In the foregoing, the oscillator 10 of the first embodiment includes first and second oscillating members 12, 14 outputting first and second centi-millimeter waves having first and second frequencies, respectively. Rather, the present invention is not limited thereto, the oscillator 10 may have three or more oscillating members. In case where three oscillating members are provided for illuminating centi-millimeter waves having frequencies different from one another, six parameters including the reflection powers (Γ₁, Γ₂, Γ₃) and the reflection phases (Φ₁, Φ₂₁, Φ₃) of the specimen are measured for another correction function expressed by a quadratic equation with six unknown parameters, thereby to estimate the blood-sugar level (BS) in an even more precise manner. In this regard, although more precise estimation of the blood-sugar level (BS) can be expected if the centi-millimeter waves having more frequencies different from one another are illuminated (i.e., the correction function has more parameters of the reflection powers and the reflection phases), the processor 50 has to take more burden of computational complexity (calculation amount) accordingly.

Modification 2.

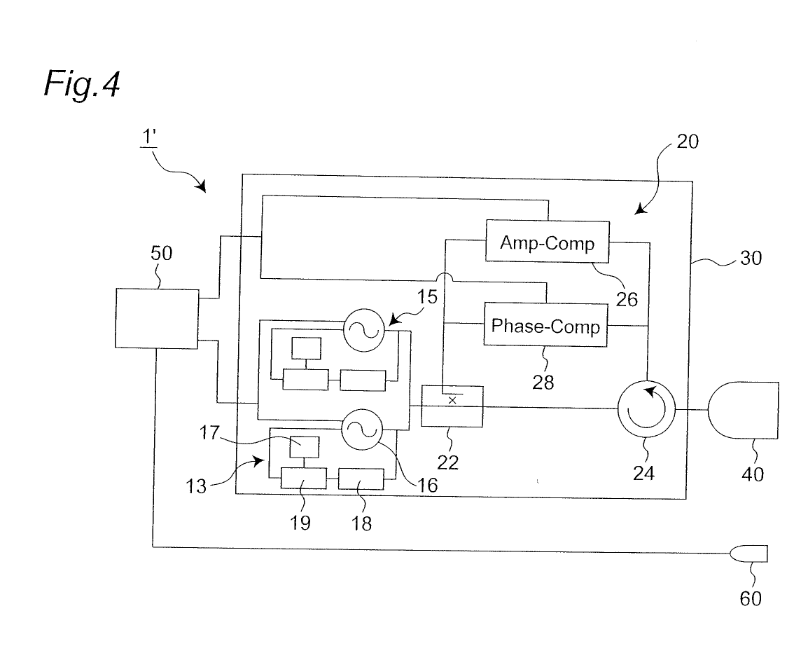

Also, in addition to the first and second oscillating members 12, 14 of the oscillator 10 according to the first embodiment, the measuring system 1′ of Modification 2 may further include first and second phase-synchronizing loop circuitries 13, 15 for stabilizing the frequencies of signals output from the oscillating members 12, 14, respectively, as illustrated in FIG. 4. Each of the first and second phase-synchronizing loop circuitries 13, 15 includes a voltage-control oscillating element 16, an internal oscillating element 17, a frequency dividing element 18, and a phase comparing element 19. The voltage-control oscillating element 16 oscillates at variable frequencies based upon a voltage on a voltage-control terminal. The internal oscillating element 17 outputs a reference input signal. The frequency dividing element 18 divides a signal output from the voltage-control oscillating element 16 into a lower frequency signal. The phase comparing element 19 compares the lower frequency signal from the frequency dividing element 18 with the reference input signal from the internal oscillating element 17 to output (feedback) a voltage signal in accordance with the phase shift therebetween, to the voltage-control terminal of the voltage-control oscillating element 16. Thus, the measuring system 1′ of Modification 2 can reduce a noise of the phase shift on the voltage-control oscillating element 16 by means of the first and second phase-synchronizing loop circuitries 13, 15, for more precise measurement of the reflection phase (Φ), thereby to estimate the blood-sugar level (BS) in a more reliable manner.

Modification 3.

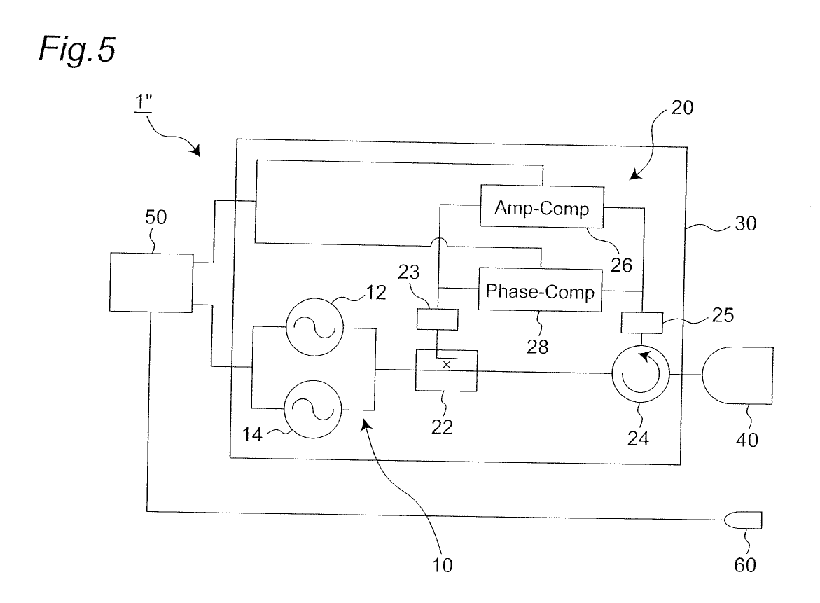

Further, according to the detector 20 of the first embodiment, the amplitude comparator 26 and the phase comparator 28 are connected directly with the coupler 22 and the circulator 24. Meanwhile, as illustrated in FIG. 5, the measuring system 1″ of Modification 3 may include a first frequency dividing element 23 intervened between the coupler 22 and the amplitude comparator 26 (and the phase comparator 28), and a second frequency dividing element 25 intervened between the circulator 24 and the amplitude comparator 26 (and the phase comparator 28). Thus, the measuring system 1″ of Modification 3 divides the signals output from the oscillating members 12, 14 and the signal reflected at the specimen into lower frequency signals, for more precise measurement of the reflection powers (Γ) and the reflection phase (Φ), thereby to estimate the blood-sugar level (BS) in an even more reliable manner.

Modification 4.

While the cavity resonator 40 of the first embodiment has a function as resonating two centi-millimeter waves having the first and second frequencies different from each other, it may be embodied in various structures as described hereinafter.

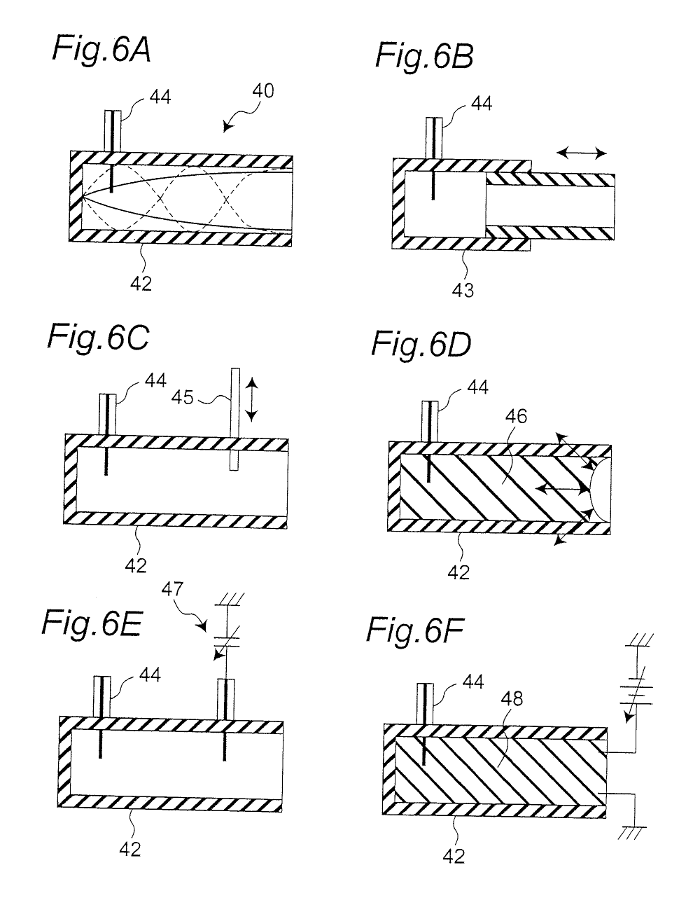

The cavity resonator 40 shown in FIG. 6A includes a hollow chassis 42 and a coaxial cable 44 extending from the oscillation-detection apparatus 30 and being inserted within the chassis 42 at the end thereof, which is sized to resonate at least two, and preferably more of centi-millimeter waves having different frequencies.

The cavity resonator 40 shown in FIG. 6B includes a structure similar to that of FIG. 6B, and includes a telescopic chassis 43 of which length along a longitudinal direction (traveling direction of the centi-millimeter waves) can be adjusted. Thus, the centi-millimeter waves of any frequencies can be resonated by adjusting the length of the telescopic chassis 43 of the cavity resonator 40.

The cavity resonator 40 shown in FIG. 6C uses a dielectric rod 45 adjustably inserted within the chassis 42 at the other end thereof, of which insertion length can be adjusted for controlling the electrical length and thus the resonating frequency of the cavity resonator 40.

The cavity resonator 40 shown in FIG. 6D includes a dielectric material 46 filled within the chassis 42, of which configuration can be tuned to control the electrical length and thus the resonating frequency.

The cavity resonator 40 shown in FIG. 6E uses a phase shifter 47 inserted within the chassis 42 at the other end thereof, of which control voltage can be modulated to control the electrical length and thus the resonating frequency.

The cavity resonator 40 shown in FIG. 6F includes a dielectric material 48 filled within the chassis 42, of which dielectric constant can be modified by a voltage applied thereto, thereby to adjust the electrical length and thus the resonating frequency.

### Embodiment 2

Next, a second embodiment of the measuring system according to the present invention will be described herein. The measuring system 2 of the second embodiment has a structure similar to that of the first embodiment except that a complex permittivity (relative permittivity) of the specimen is used, rather than the reflection coefficient, to estimate the serum glucose concentration. Therefore, duplicate description is eliminated for the similar structure. Like reference numerals are used for like components for the present embodiment.

In general, the reflection coefficient (R) can be expressed by the reflection power (Γ) and the reflection phase (Φ) in the following equation.

R=Γ×exp(i×Φ)

wherein “i” is an imaginary unit.
Also, the complex permittivity (∈) can be expressed as a function of the reflection coefficient (R).

∈=F(R)

Thus, the complex permittivity (∈) can be calculated by measuring the reflection power (Γ) and the reflection phase (Φ). Therefore, as the reflection power (Γ) and the reflection phase (Φ) has a frequency dependency varying with frequency of the centi-millimeter wave, the complex permittivity (∈) also has a frequency dependency varying in accordance with the frequency of the centi-millimeter wave.

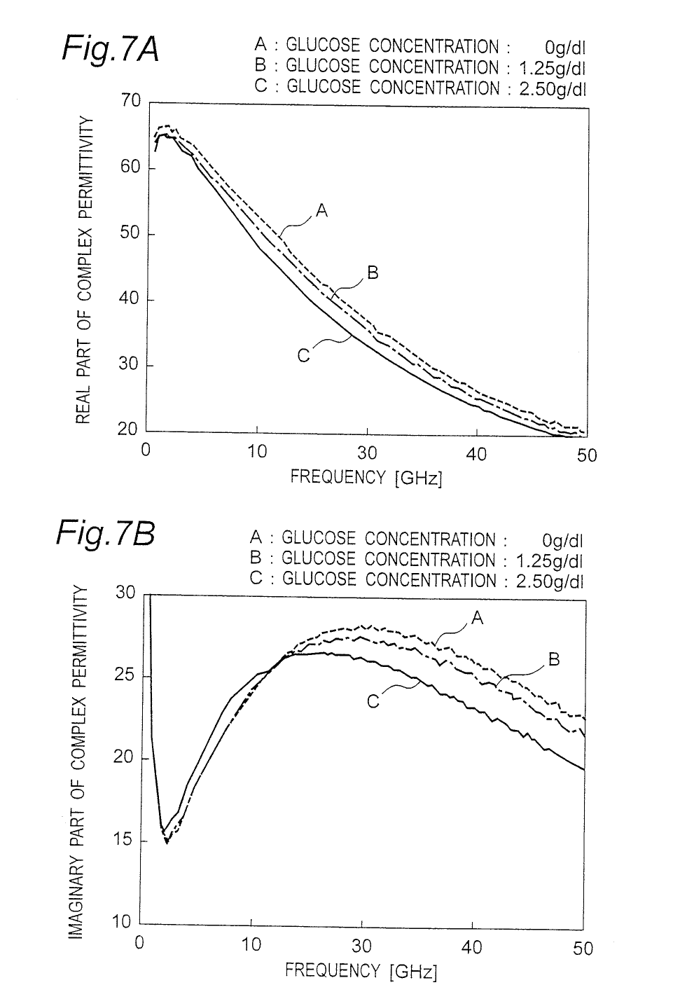

FIGS. 7A and 7B are charts illustrating real and imaginary parts of the complex permittivity (∈), respectively, calculated from the reflection power and the reflection phase of the blood which are measured upon illumination of the centi-millimeter waves having frequency of 1 GHz through 40 GHZ. In particular, those graphs show the real and imaginary parts of the complex permittivity (∈) of the blood containing different serum glucose concentrations of 0 g/dl (A), 1.25 g/dl (B), and 2.50 g/dl (C). As can be seen in FIGS. 7A and 7B, the real and imaginary parts of the complex permittivity (∈) show a different frequency dependency due to the serum glucose concentration.

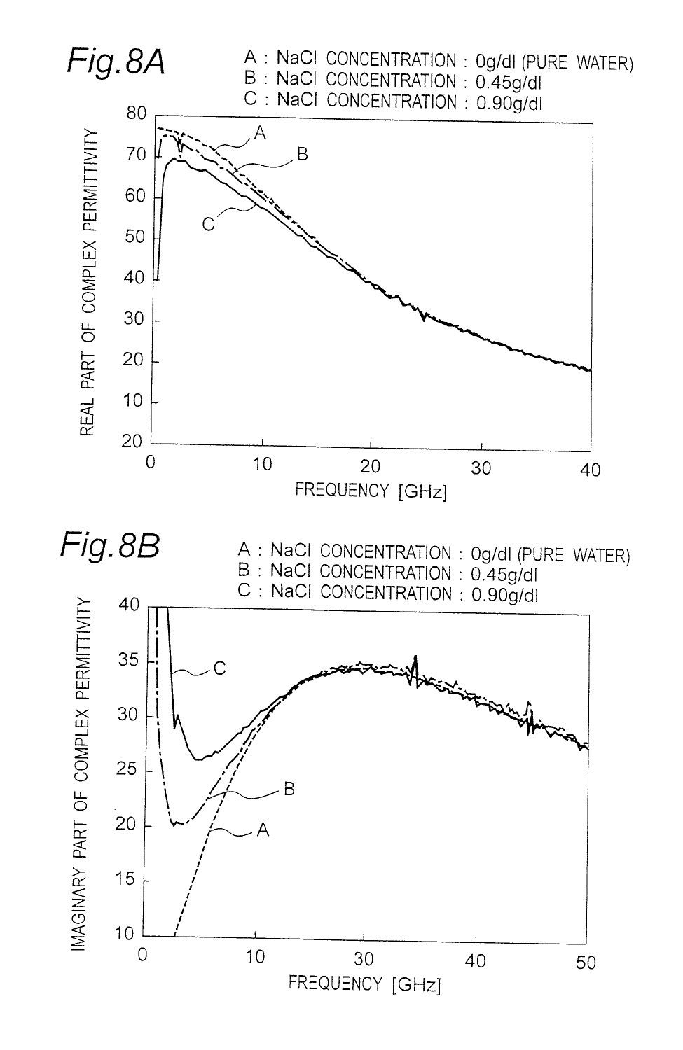

Similarly, FIGS. 8A and 8B are graphs plotting real and imaginary parts of the complex permittivity (∈), respectively, of water having different sodium chloride concentrations of 0 g/dl (A, pure water), 0.45 g/dl (B), and 0.90 g/dl (C). Thus, as shown in FIGS. 8A and 8B, the real and imaginary parts of the complex permittivity (∈) show a different frequency dependency also due to the sodium chloride concentration.

The blood contains the sodium chloride, of which concentrations may substantially change due to subject's drinking (and eating) and sweating. As the measuring system 2 of the present invention is to precisely measure the serum glucose concentration, the influence of the sodium chloride concentration should be minimized. Again referring to FIGS. 8A and 8B, the real part of the complex permittivity is rapidly reduced at frequency of 1 GHz or less, while the imaginary part of the complex permittivity is rapidly increased at frequency of 1 GHz or more. In other words, the measurement of the complex permittivity with the centi-millimeter wave having frequency of 5 GHz or more reduces the influence of the sodium chloride concentration for the measured complex permittivity. Thus, it is preferable to use the centi-millimeter wave having frequency of 5 GHz or more, for measuring the complex permittivity (∈) (reflection coefficient (R)). Also, it has been proved quite difficult to precisely measure the complex permittivity (∈) (reflection coefficient (R)) when using a currently available, versatile oscillation-detection apparatus illuminating a sub-millimeter wave of 300 GHz or more that is higher than the centi-millimeter wave. Therefore, in order to achieve the precise measurement with the oscillation-detection apparatus 30 that is available at a relatively reasonable cost, the centi-millimeter wave having frequency of 300 GHz or less is advantageously used for precisely measuring the complex permittivity (∈) (reflection coefficient (R)). Therefore, according to the present invention, in particular, the centi-millimeter wave of frequency between 5 GHz and 300 GHz is advantageously used for precise measurement of the complex permittivity (∈).

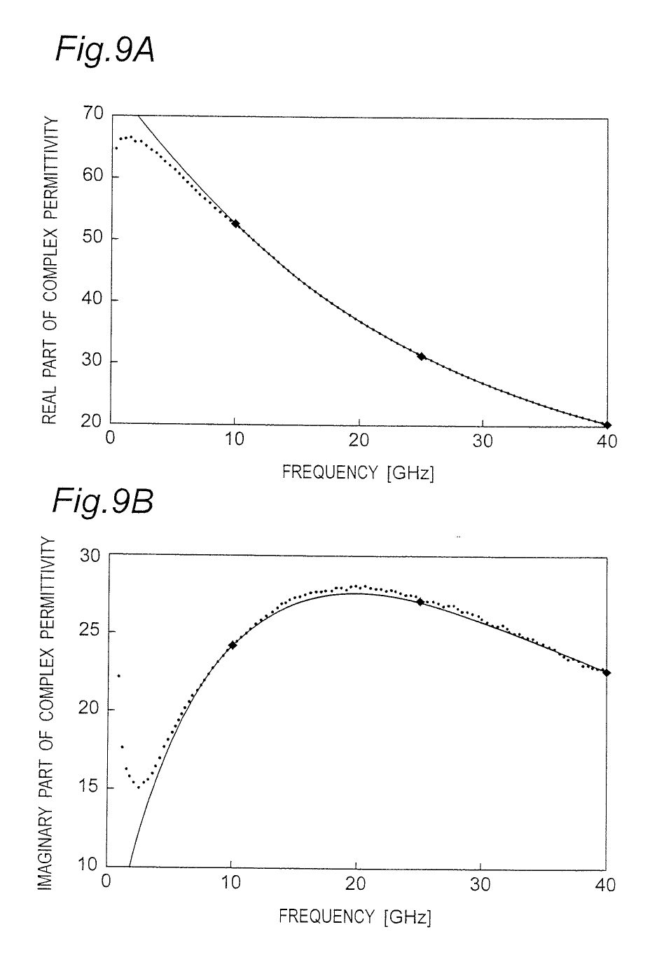

FIGS. 9A and 9B are charts illustrating with discrete dots, a plurality (about a hundred) of measured points of real and imaginary parts of the complex permittivity of millimeter waves, respectively, which are measured upon illumination of the centi-millimeter waves having various frequencies between 5 GHz and 300 GHz towards the specimen.

Meanwhile, it is known that the complex permittivity (∈) can generally be approximated by various dielectric relaxation equations with a variable (parameter) of frequency (f), and for example, the Harvriliak-Negami dielectric relaxation equation can be adapted for fitting the measured real and imaginary parts of the complex permittivity. Thus, the measured real and imaginary parts of the complex permittivity can be fit with appropriate factors of the Harvriliak-Negami equation for continuous approximation. Thus, FIGS. 9A and 9B illustrate such continuous approximation of the real and imaginary parts of the complex permittivity, respectively, as trajectories of the dielectric relaxation equation, together with the discrete measured dots thereof.

$$ɛ ( f ) = ɛ ( ∞ ) + \frac{ɛ ( 0 ) - ɛ ( ∞ )}{{\{ 1 + {( i f / {f}_{0} )}^{β} \}}^{α}}$$

### <Harvriliak-Negami Dielectric Relaxation Equation>

The parameter (f) represents frequency, and the function ∈(f) expresses the complex permittivity at frequency of (f). Also, ∈(0) is the real part of the complex permittivity at frequency of zero, ∈(∞) is the real part of the complex permittivity at frequency of infinite, (f₀) is a peak frequency of the imaginary part of the complex permittivity, and (α) and (β) are correction factors, all of which are real fitting factors of the equation.

Besides the above dielectric relaxation equation, there are other following dielectric relaxation equations known as the Debye dielectric relaxation equation, the Davidson-Cole dielectric relaxation equation, and the Cole-Cole dielectric relaxation equation. Each of those dielectric relaxation equations has a set of fitting factors as listed below, used for fitting the real and imaginary parts of the complex permittivity therewith, which are measured with several waves at frequency between 4 GHz and 40 GHz for the blood containing the serum glucose concentration, for example, 2.5 g/dl.

$$ɛ ( f ) = ɛ ( ∞ ) + \frac{ɛ ( 0 ) - ɛ ( ∞ )}{1 + i f / {f}_{0}}$$

### <Debye Dielectric Relaxation Equation>

$$ɛ ( f ) = ɛ ( ∞ ) + \frac{ɛ ( 0 ) - ɛ ( ∞ )}{{( 1 + i f / {f}_{0} )}^{α}}$$

### <Davidson-Cole Dielectric Relaxation Equation>

$$ɛ ( f ) = ɛ ( ∞ ) + \frac{ɛ ( 0 ) - ɛ ( ∞ )}{1 + {( i f / {f}_{0} )}^{β}}$$

### <Cole-Cole Dielectric Relaxation Equation>

TABLE 3

| Dielectric | | | | | | Mean |
|---|---|---|---|---|---|---|
| Relaxation Equation | ε (∞) | ε (0) | f0 | α | β | Error |
| Debye | 11.55 | 65.77 | 15.98 | — | — | 3.4 |
| Davidson-Cole | −2.05 | 68.11 | 9.35 | 0.56 | — | 1.2 |
| Cole-Cole | 5.23 | 72.56 | 15.38 | — | 0.85 | 0.6 |
| Harvriliak-Negami | 14.31 | 78.57 | 80.16 | 3.35 | 0.69 | 0.2 |

As above, in the measuring system 2 according to the second embodiment, the oscillation-detection apparatus 30 measures the complex permittivity at several points of frequency, and the processor 50 fits the measured discrete data with the dielectric relaxation equation, thereby to characterize the polarization property (dielectric property) of the specimen as a set of fitting factors, i.e., ∈(0), ∈(−), f₀, α and β. Thus, the fitting factors of the dielectric relaxation equation define the dielectric property of the specimen and the constituent concentration thereof (the concentration of serum glucose concentration).

Therefore, according to the second embodiment, similar to the first embodiment, the processor 50 presumes a correction function expressed by a quadratic equation with multiple unknown parameters of each of the fitting factors, for determining the blood-sugar level (BS). For example, when the fitting factors of the Harvriliak-Negami dielectric relaxation equation are used, the blood-sugar level (BS) is presumed to be obtained as a correction function expressed by the following quadratic equation with five unknown parameters.

$$BS = {{\sum }^{5}}_{i = 1} ( {p}_{i} \times {c}_{i}^{2} + {q}_{i} \times {c}_{i} ) + s$$

In this formula, the parameters (cᵢ) represent each of five fitting factors of the dielectric relaxation equation, i.e., ∈(0), ∈(∞), f₀, α and β (“i” is an integer between 1-5 for the Harvriliak-Negami dielectric relaxation equation), and also factors (pᵢ), (qᵢ), and (s) represent factors of the correction function.

The processor 50 calculates, in advance, the correction function factors (pᵢ), (qᵢ), and (s) based upon the relationship between known serum glucose concentrations and the fitting factors of the dielectric relaxation equation therefor, which are stored in a memory (not shown) of the processor. Then, for an actual measurement, the processor 50 assigns the fitting factors of the dielectric relaxation equation obtained based upon the measured complex permittivity, into the correction function so as to precisely estimate the blood-sugar level (BS).

Although FIGS. 9A and 9B illustrate the real and imaginary parts of the complex permittivity measured for about a hundred of the centi-millimeter waves having different frequencies, the fitting factors of the dielectric relaxation equation can be determined also by illuminating the centi-millimeter waves having at least two and preferably three or more of different frequencies. The correction function factors of the first embodiment are dependent upon the frequency of the centi-millimeter wave used for the measurement of the reflection power (Γ) and the reflection phase (Φ). However, since the correction function factors of the second embodiment is independent on the frequency of the centi-millimeter wave, the measuring system 2 of the second embodiment is not required to stabilize the frequency of the centi-millimeter wave in a strict manner. Therefore, this allows a simpler structure of the measuring system 2 that can be produced at a more reasonable cost, still achieving the precise estimation of the serum glucose concentration by measuring the complex permittivity (reflection coefficient).

Modification 5.

In the foregoing, the measuring system 2 of the second embodiment is described as measuring the serum glucose concentration, the present invention can be applied to measure any other constituent concentration.

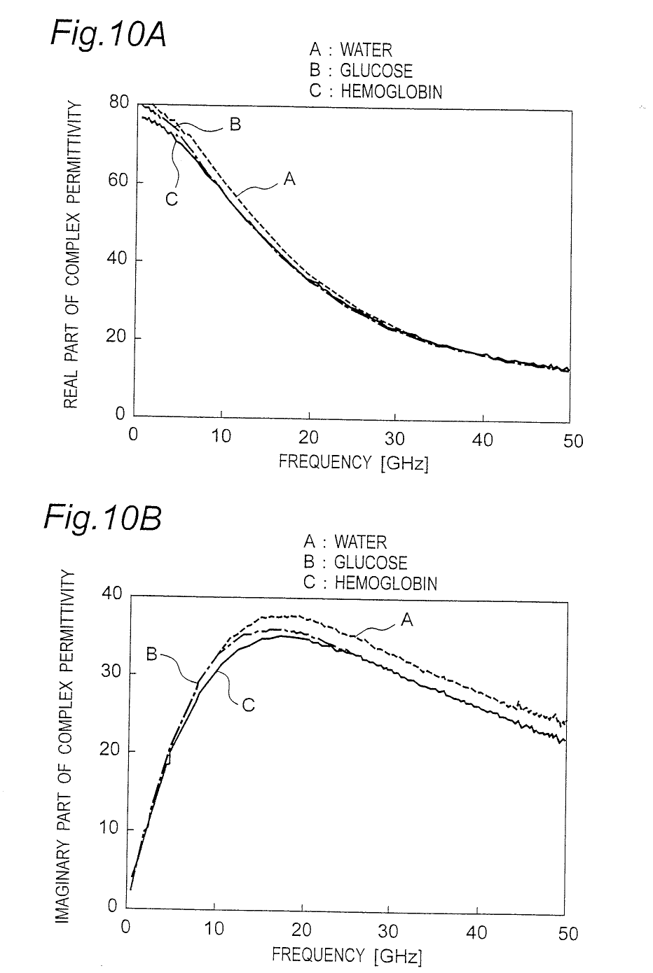

FIGS. 10A and 10B are charts illustrating a frequency dependency (permittivity property) of real and imaginary parts of the complex permittivity, respectively, of a blood containing a given amount of glucose and hemoglobin. As will be clear from FIGS. 10A and 10B, both of the real and imaginary parts of the complex permittivity (∈) are affected by concentrations of the constituents of glucose and hemoglobin contained in the blood. Therefore, the desired constituent such as hemoglobin in the blood can also be determined by means of the process similar to the second embodiment.

As described above, prior to actual measurement, the complex permittivity of the desired constituent is sampled with the centi-millimeter waves at a plurality of frequencies, and is fit with the dielectric relaxation equation to characterize the polarization property (dielectric property) of the blood containing the desired constituent as a set of fitting factors. Then, it is presumed that the concentration of the desired constituent can be expressed by the correction function in a form of a quadratic equation with multiple unknown parameters of each of the fitting factors, of which correction function factors are determined in advance. After actual measuring the complex permittivity of the specimen with the centi-millimeter waves at several frequencies,

the measured complex permittivity is assigned into the pre-defined correction function with known factors, so as to estimate the concentration of the hemoglobin in the blood.

Although estimating the concentration of the hemoglobin in the blood is discussed above in this modification, the measurement system 1, 2 according to the present invention can be used for estimating the concentration of not only the glucose and hemoglobin but also any other constituents in the blood such as γ-GTP, cholesterol, uric acid, and urea.

In addition, the complex permittivity (∈) is determined by measuring the reflection coefficient (R), i.e., the reflection power (Γ) and the reflection phase (Φ) in the second embodiment, it may be measured by any other approaches which are commonly known by a person skilled in the art. For example, the permeability coefficient (T) instead of the reflection coefficient (R) may be used for determining the complex permittivity (∈).

Further, while the first and second embodiments state user's finger as an exemplary subject to be measured, which is not limited thereto, the measurement system according to the present invention can be adapted to any other subjected portions such as an earlobe, and even also to an animal. Moreover, the measurement system according to the present invention can be used to measure the constituent concentration of fluid sample received in a test tube in a non-contact manner.

1. A system for measuring concentration of a constituent of a specimen, comprising:
an oscillator for outputting, towards the specimen, a plurality of electromagnetic waves having different frequencies in a range from 5 GHz to 300 GHz;
a detector for detecting the electromagnetic waves that are reflected from the specimen; and
a processor for measuring at least one of reflection coefficient and complex permittivity of the electromagnetic waves that are detected and calculating the concentration of the constituent of the specimen based upon at least one of the reflection coefficient measured and the complex permittivity measured.

2. The system according to claim 1, wherein
the plurality of the electromagnetic waves includes first and second electromagnetic waves respectively having first and second frequencies that are different from each other, and
said processor calculates the concentration C of the constituent of the specimen in accordance with a correction function having parameters of reflection powers Γ₁ and Γ₂, and reflection phases Φ₁ and Φ₂, of the reflection coefficient measured, as
C=a×Γ₁²+b×Γ₁+c×Φ₁²+d×Φ₁+e×Γ₂²+f×Γ₂+g×Φ₂²+h×Φ₂+i, and
“a” through “i” are constants.

3. The system according to claim 2, wherein said processor determines the complex permittivity of the specimen for a plurality of the electromagnetic waves based upon a plurality of the reflection powers measured and the reflection phases measured.

4. The system according to claim 1, further comprising a cavity resonator connected to said oscillator and said detector, said cavity resonator contacting the specimen.

5. The system according to claim 4, wherein said cavity resonator has a plurality of resonant frequencies.

6. The system according to claim 1, wherein said processor determines a plurality of parameters of an approximation formula which continuously defines a relationship between the frequency of the electromagnetic waves and corresponding complex permittivity, and calculates the concentration of the constituent of the specimen based upon the parameters of the approximation formula.

7. The system according to claim 6, wherein
the approximation formula is expressed by one equation selected from the group consisting of the Debye dielectric relaxation equation, the Davidson-Cole dielectric relaxation equation, the Cole-Cole dielectric relaxation equation, and the Harvriliak-Negami dielectric relaxation equation, which are, respectively
$$ɛ ( f ) = ɛ ( ∞ ) + \frac{ɛ ( 0 ) - ɛ ( ∞ )}{1 + i f / {f}_{0}} ,$$
$$ɛ ( f ) = ɛ ( ∞ ) + \frac{ɛ ( 0 ) - ɛ ( ∞ )}{( 1 + {( i f / {f}_{0} )}^{α}} ,$$
$$ɛ ( f ) = ɛ ( ∞ ) + \frac{ɛ ( 0 ) - ɛ ( ∞ )}{{( 1 + i f / {f}_{0} )}^{β}} , and$$
$$ɛ ( f ) = ɛ ( ∞ ) + \frac{ɛ ( 0 ) - ɛ ( ∞ )}{{\{ 1 + {( i f / {f}_{0} )}^{β} \}}^{α}} ,$$
and
f is frequency, ∈(0) is the real part of the complex permittivity at zero frequency, ∈(∞) is the real part of the complex permittivity at infinite frequency, f₀ is peak frequency of the imaginary part of the complex permittivity, and α and β are correction factors, which are real fitting factors.

8. The system according to claim 6, wherein said processor expresses the concentration of the constituent as a correction function with regard to the parameters of the approximation formula, determines factors of the correction function in advance, and assigns the parameters of the approximation formula that are measured to estimate the concentration of the constituent.

9. The system according to claim 1, wherein
the specimen is a biological body, and
the constituent contained within the specimen is at least one selected from the group consisting of glucose, γ-GTP, hemoglobin, cholesterol, albumin, uric acid, and urea.

10. A method for measuring concentration of constituent of a specimen, comprising:
outputting, towards the specimen, a plurality of electromagnetic waves having different frequencies in a range from 5 GHz to 300 GHz;
detecting the electromagnetic waves that are reflected from the specimen; and
measuring at least one of reflection coefficient and complex permittivity of the electromagnetic waves that are detected; and
calculating the concentration of the constituent of the specimen based upon at least one of the reflection coefficient measured and the complex permittivity measured.

11. The method according to claim 10, wherein
the plurality of the electromagnetic waves includes first and second electromagnetic waves respectively having first and second frequencies that are different from each other, and
calculating the concentration C of the constituent of the specimen in accordance with a correction function having parameters of reflection powers Γ₁ and Γ₂, and reflection phases Φ₁ and Φ₂, of the reflection coefficient measured, as
C=a×Γ₁²+b×Γ₁+c×Φ₁²+d×Φ₁+e×Γ₂²+f×Γ₂+g×Φ₂²+h×Φ₂+i, and
“a” through “i” are constants.

12. The method according to claim 11, further comprising determining the complex permittivity of the specimen for a plurality of the electromagnetic waves based upon a plurality of the reflection powers measured and the reflection phases measured.

13. The method according to claim 10, wherein calculating the concentration of the constituent of the specimen includes,
determining a plurality of parameters of an approximation formula which continuously defines a relationship between the frequency of the electromagnetic waves and corresponding complex permittivity, and
calculating the concentration of the constituent of the specimen based upon the parameters of the approximation formula.

14. The method according to claim 13, wherein
the approximation formula is expressed by one equation selected from the group consisting of the Debye dielectric relaxation equation, the Davidson-Cole dielectric relaxation equation, the Cole-Cole dielectric relaxation equation, and the Harvriliak-Negami dielectric relaxation equation, respectively
$$ɛ ( f ) = ɛ ( ∞ ) + \frac{ɛ ( 0 ) - ɛ ( ∞ )}{1 + i f / {f}_{0}} ,$$
$$ɛ ( f ) = ɛ ( ∞ ) + \frac{ɛ ( 0 ) - ɛ ( ∞ )}{( 1 + {( i f / {f}_{0} )}^{α}} ,$$
$$ɛ ( f ) = ɛ ( ∞ ) + \frac{ɛ ( 0 ) - ɛ ( ∞ )}{{( 1 + i f / {f}_{0} )}^{β}} , and$$
and
$$ɛ ( f ) = ɛ ( ∞ ) + \frac{ɛ ( 0 ) - ɛ ( ∞ )}{{\{ 1 + {( i f / {f}_{0} )}^{β} \}}^{α}} ,$$
f is frequency, ∈(0) is the real part of the complex permittivity at zero frequency, ∈(∞) is the real part of the complex permittivity at infinite frequency, f₀ is peak frequency of the imaginary part of the complex permittivity, and α and β are correction factors, which are real fitting factors.

15. The method according to claim 14, including
expressing the concentration of the constituent as a correction function with regard to the parameters of the approximation formula,
determining factors of the correction function in advance,
assigning the parameters of the approximation formula that are measured, and
estimating the concentration of the constituent.

16. The method according to claim 10, wherein
the specimen is a biological body, and
the constituent contained within the specimen is at least one selected from the group consisting of glucose, γ-GTP, hemoglobin, cholesterol, albumin, uric acid, and urea.
